(19) United States
(12) Patent Application Publication
(10) Pub. No.: US 2010/0029602 A1
(43) Pub. Date: Feb. 4, 2010

(54) LOW-DOSE MOMETASONE FORMULATIONS

(75) Inventors: Moshe Arkin, Kfar Shmaryahu (IL); Amira Zeevi, Omer (IL); Stephen Cherkez, Caesarea (IL); Eilon Asculai, Lehavim (IL); Rina Uzan, Beer Sheva (IL); Bella Braghinski, Beer Sheva (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Perrigo Israel Pharmaceuticals Ltd., Bnei Brak (IL)

(21) Appl. No.: 12/535,406

(22) Filed: Aug. 4, 2009

Related U.S. Application Data
(63) Continuation-in-part of application No. PCT/IL2008/000485, filed on Apr. 9, 2008.
(60) Provisional application No. 60/911,185, filed on Apr. 11, 2007.

(51) Int. Cl. A61K 31/58; A61P 17/02; A61P 17/06

(52) U.S. Cl. 514/172

(57) ABSTRACT

Provided is a topical cream composition for the delivery of mometasone furoate comprising low dose mometasone furoate for the treatment of corticosteroid responsive dermatoses. The composition of the present invention can be safely applied over large surface areas of the skin (including areas with wrinkles and/or hair), and can be used therapeutically for extended periods of time (e.g., greater than 3 weeks). Treatment with the composition of the present invention carries reduced and/or fewer side effects compared with commercially available mometasone furoate cream products. The cream composition of the present invention is safe for the use of babies and infants under 2 years old. Additionally provided are methods of preparing and using the composition of the invention.

### CROSS-REFERENCE TO RELATED APPLICATIONS

This patent application is a continuation-in-part of copending International Application No. PCT/IL2008/000485, filed Apr. 9, 2008, which is incorporated by reference, and claims the benefit of U.S. Provisional Patent Application No. 60/911,185, filed Apr. 11, 2007, which is incorporated by reference.

### BACKGROUND OF THE INVENTION

Mometasone furoate is a corticosteroid compound that is useful as a topical anti-inflammatory compound. See, e.g., U.S. Pat. No. 4,472,393 (“the '393 patent”), which discloses topical compositions that include, inter alia, mometasone furoate for the treatment and control of inflammatory conditions. Mometasone furoate is practically insoluble in water, slightly soluble in octanol, and moderately soluble in ethyl alcohol.

Mometasone furoate cream is currently manufactured and sold as a topical cream under the trademark Elocon® Cream 0.1%. According to the product label, each gram of Elocon® Cream 0.1% contains mometasone furoate, USP (1 mg) in a cream base of hexylene glycol, NF; phosphoric acid, NF; propylene glycol stearate (55% monoester); stearyl alcohol and ceteareth-20; titanium dioxide, USP; aluminum starch octenylsuccinate (Gamma Irradiated); white wax, NF; white petrolatum, USP; and purified water, USP.

Elocon® Cream 0.1% is indicated for the relief of the inflammatory and pruritic manifestations of corticosteroid-responsive dermatoses. According to the prescribing information, however, Elocon® Cream 0.1% caused HPA axis suppression in approximately 16% of pediatric patients ages 6 to 23 months and is not recommended for patients under 2 years of age. Although the prescribing information for Elocon® Cream 0.1% indicates that it may be used “with caution” in patients 2 years of age or older, the prescribing information also warns that pediatric patients may be more susceptible to systemic toxicity, including HPA axis suppression and Cushing's syndrome due to their larger skin surface to body mass ratios. Pediatric patients also are at greater risk of adrenal insufficiency during and/or after withdrawal of treatment, and skin atrophy. The prescribing information for Elocon® Cream 0.1% also states that pediatric patients applying topical corticosteroids to greater than 20% of body skin area are at greater risk of HPA axis suppression.

Elocon® Cream 0.1% (like most conventional oil-based-containing topical corticosteroid creams) is formulated with relatively low concentrations of water. For instance, U.S. Pat. No. 4,808,610, which describes Elocon® Cream 0.1%, teaches formulating mometasone furoate in a vehicle that contains relatively low concentrations of water (1.0 to 5.0 percent). Elocon® Cream 0.1% tends to have an oily texture and tends to be rather difficult to wash off the skin. Oily compositions like Elocon® Cream 0.1% also can become tacky or sticky. Nevertheless, oily compositions are used conventionally to provide the occlusiveness needed for topical efficacy. See, e.g., McKenzie A W, et al., Arch Dermatol 86:608-10 (1962), which teaches the importance of hydration on skin in cortico steroid therapy as demonstrated by employing an occlusive plastic film. The oily and/or adherent properties of Elocon® Cream 0.1% may discourage patient compliance with the prescribed treatment regimen.

As such, there is a need for topical mometasone furoate compositions that are relatively non-toxic, yet safe and effective for treating corticosteroid-responsive skin conditions, particularly in pediatric patients under 2 years of age. There is also need for topical mometasone furoate compositions that can be safely applied over large surface areas of the skin (e.g., greater than 20% of the total skin surface) and/or for a significant duration of time (e.g., greater than 3 weeks), with reduced side effects and toxicity as compared to treatment with Elocon® Cream 0.1%. There is also a need for cosmetically elegant mometasone furoate compositions that are pleasant, easy to apply, leave fewer residues, are easy to wash off, and less adherent or irritating to damaged skin. The present invention provides such compositions, and methods for preparing and using such compositions.

### BRIEF SUMMARY OF THE INVENTION

The present invention provides a unique, elegant, safe and effective composition for the topical delivery of mometasone furoate. The composition of the present invention includes from about 0.05 wt % to less than 0.1 wt % mometasone furoate (e.g., from about 0.075 wt % to less than 0.1 wt % mometasone furoate) and a pharmaceutically acceptable vehicle that includes an oily phase and at least about 30 wt % water (e.g., from about 30 wt % to about 65 wt % water). Preferably, the composition further includes a polyol (which can include one or more polyols) and a gelling agent (which can include one or more gelling agents). In a preferred embodiment, the composition of the present invention is formulated as a topical cream. Exemplary compositions of the present invention include about 0.075 wt % mometasone furoate, a polyol, a gelling agent, at least about 30 wt % water (e.g., about 60 wt % water), and an oily phase (e.g., about 20 wt % of an oily phase).

The composition of the present invention is effective for treating corticosteroid responsive dermatoses. The composition of the present invention can be safely applied over large surface areas of the skin (including areas with wrinkles and/or hair), and can be used therapeutically for extended periods of time (e.g., greater than 3 weeks).

Moreover, treatment with the composition of the present invention carries reduced and/or fewer side effects compared with treatment with Elocon® Cream. Side effects that are decreased and/or avoided by the compositions of the present invention can include steroid rosacea, atrophy (skin thinning) and striae (stretch marks), easy bruising and tearing of the skin, perioral dermatitis (rash around the mouth), telangiectasia (enlarged blood vessels), susceptibility to skin infections, tinea incognito (disguising infection). Withdrawal symptoms that have been associated with cessation of mometasone treatment, including glucocorticosteroid insufficiency, can also be reduced or avoided. The compositions of the present invention are associated with decreased HPA axis suppression, a common side effect of Elocon® Cream which can lead to increased risk of Cushing's syndrome, hyperglycemia, and glucosuria.

Without wishing to be bound by any particular theory, it is believed that the composition of the present invention has a relatively low systemic steroid absorption, as compared with Elocon® Cream.

In addition, the composition of the present invention has a pleasant texture, is easy to apply, leaves fewer residues on the treated area after application, is much easier to wash off and remove from the skin, and is less tacky and less adherent to damaged skin than conventional oily compositions, yet provides the occlusiveness needed for effective topical therapy.

Without wishing to be bound by any particular theory, it is believed that the composition of the present invention contains a relatively high percentage of “entrapped” or “bound” water in the continuous phase, which is water that is bound to hydrophilic groups and counterions. Bound water tends to exhibit a phase-transition temperature lower than that of bulk water due to the associative interactions with other components in the composition (for example, with the hydrophilic group of polymers). Bound water typically melts at a temperature lower than −10° C. This temperature normally decreases as function of the association strength for bound water, the molecules of which are entrapped or bound, and not readily free to engage in the types of interactions normally exhibited by free water. By contrast, free water in an emulsion system tends to have properties similar to those of pure (bulk) water. Free water normally melts (or freezes—depending on the applied method) at 0° C. and is characterized by transition enthalpy (AH) of −324 J/g. “Interphasal” water is confined within the region separating the aqueous core from the oleic dispersing phase. This region is considered to be finite in extent. Interphasal water typically melts at temperatures between −5° C. and −10° C.

The present invention additionally provides a method for producing a cream composition, which preferably includes mixing an active phase with an oily phase; combining a water phase with the mixture of oily and active phases, and homogenizing the phases to produce a substantially uniform dispersion; optionally adjusting the pH to obtain a pH of from about 4.0 to about 5.5; and, optionally, further diluting with water, to produce the cream composition. The active phase preferably includes from about 0.05 wt % to less than 0.1 wt % mometasone furoate (e.g., from about 0.075 wt % to less than 0.1 wt % mometasone furoate) and a polyol, the oily phase preferably includes a fatty acid, a fatty alcohol, and a fatty acid ester, and the water phase preferably includes one or more gelling agents. The composition prepared in accordance with the method of the present invention preferably includes at least about 20 wt % water (e.g., from about 30 wt % water to about 65 wt % water, e.g., about 60 wt % water).

The present invention further provides a method for treating a corticosteroid-responsive disease or condition associated with a patient's skin, which method includes topically applying to the patient (e.g., on the patient's skin in or near the affected area) the composition of the present invention in an amount effective to treat the disease or condition. The disease or condition can include, e.g., corticosteroid-responsive dermatoses, atopic dermatitis, and the like. In a preferred embodiment, the method of the present invention includes topically treating atopic dermatitis in pediatric patients under 2 years of age with reduced side effects as compared to treatment with Elocon® Cream, e.g., decreased HPA axis suppression.

### BRIEF DESCRIPTION OF THE SEVERAL VIEWS OF THE DRAWING(S)

FIG. 1 graphically depicts the results of a comparative penetration test.

FIG. 2 graphically depicts the results of a comparative penetration test.

FIG. 3 graphically depicts the results of a histopathological analysis test.

### DETAILED DESCRIPTION OF THE INVENTION

The composition of the present invention can be administered topically using methods that are well known in the art, e.g., by directly applying or spreading the composition on an affected area, which can include surfaces of the skin, and the like.

The composition of the present invention preferably includes from about 0.05 wt % to less than 0 1 wt % mometasone furoate, e.g., from about 0.075 wt % to less than 0.1 wt % mometasone furoate (e.g., about 0.075 wt % mometasone furoate), and a pharmaceutically acceptable vehicle that includes an oily phase and at least about 20 wt % water (e.g., from about 30 wt % to about 65 wt % water, e.g., about 60 wt % water). The vehicle is preferably chosen from components that are suitable for use in contact with mammalian skin without producing undue toxicity, incompatibility, instability, allergic responses or the like. It will be appreciated that the precise amount of mometasone furoate to be used in the composition of the present invention, the choice of vehicle components and the amount of composition to be administered to a particular patient, can vary depending on a variety of factors. Such factors may include, e.g., the nature of the skin being treated, the age and physical condition of the subject being treated, the severity of the condition, the duration of the treatment, the nature of any concurrent therapy that may apply, the nature of the active agent(s) and topical carrier(s) employed, and the like. In a preferred embodiment, the composition of the present invention includes about 0.075 wt % to less than 0.1 wt % mometasone furoate, a polyol, a gelling agent, at least about 20 wt % water, and an oily phase. Exemplary compositions of the present invention include about 0.075 wt % mometasone furoate, a polyol, a gelling agent, at least about 30 wt % water (e.g., about 60 wt % water), and an oily phase (e.g., about 15 wt % of an oily phase).

The composition of the present invention is suitable for topical administration to mammalian skin, preferably human skin. The skin can be covered with hair and/or can have wrinkles. The composition of the present invention can be used for topically treating corticosteroid-responsive skin conditions and/or diseases, such as, e.g., inflammation, psoriasis, hyperkeratotic dermatosis, pruritic manifestations of corticosteroid-responsive dermatoses, eczema, atopic dermatitis and the like.

The composition of the present invention preferably includes from about 12 wt % to about 30 wt % of a polyol. In a preferred embodiment, the composition of the present invention includes about 15 wt % of a polyol. Any polyol suitable for topical administration can be used in the composition of the present invention. Preferred polyols include propylene glycol and hexylene glycol. Other suitable polyols can include commercially available or known polyols such as, e.g., pentaerythritol, ethylene glycol, polyethylene glycol, polypropylene glycol, sorbitol, glycerol, polyglycerol, saccharides, cyclodextrins, synthetic polyhydric polymers, and the like, and combinations thereof. Preferably, the polyol used in the composition of the present invention has humectants properties (e.g., propylene glycol, hexylene glycol, sorbitol, glycerine, etc.).

The composition of the present invention preferably includes from about 0 wt % to about 0.9 wt % of one or more gelling agents. In a preferred embodiment, the composition of the present invention includes about 0.5 wt % of one or more gelling agents. Suitable gelling agents can include, e.g., pharmaceutically acceptable natural, synthetic, and semisynthetic gelling agents. Preferably, the gelling agent includes a polyacrylic acid, such as, for example, a carbomer, e.g., carbomer 910, carbomer 934, carbomer 940, carbomer 941 and the like, and combinations thereof. Other suitable gelling agents can include, e.g., carboxymethylcellulose, ethylcellulose, gelatin, hydroxyethyl cellulose, hydroxypropyl cellulose, magnesium aluminum silicate, methylcellulose, poloxamers, polyvinyl alcohol, sodium alginate, alginic acid, tragacanth, acacia, bentonite, poly(amino amine)-xanthan gum, and the like, and combinations thereof.

In some embodiments, the composition of the present invention includes two or more gelling agents. Preferred combinations of gelling agents include combinations of xanthan gum and a carbomer. In some particularly preferred formulations, the composition includes about 0.4 wt % of xanthan gum or less, and/or about 0.5 wt % of Carbomer 940 or less.

The composition of the present invention also can include a neutralizing agent, which can serve to activate the gelling agent. One skilled in the art can easily determine the appropriate neutralizing agent to be used for activating a particular gelling agent. A neutralizing agent can be any pH adjusting agent. Suitable neutralizing agents include basic neutralizing agents, which also be used to increase the pH of the composition. Suitable neutralizing agents also can include acids, as well as buffers. Exemplary neutralizing agents include sodium hydroxide, potassium hydroxide, triethanolamine, sodium phosphate dibasic, dibasic sodium phosphate heptahydrate, phosphoric acid, and the like, and combinations thereof.

The composition of the present invention preferably includes at least about 20 wt % water, e.g., at least about 30 wt % water, about 30 wt % to about 65 wt % water, about 40 wt % to about 65 wt % water, about 50 wt % to about 60 wt % water, e.g., about 60 wt % water. Preferably, at least a substantial portion of the water in the composition of the present invention is bound water. More preferably, substantially all of the water in the composition of the present invention is bound water.

The composition of the present invention can exist in any form suitable for topical administration, e.g., an oil-in-water emulsion, a liposomal suspension, a cubosomal suspension, a vesicular suspension, or the like. Preferably, the composition of the present invention exists as an oil-in-water emulsion.

The composition of the present invention can further include one or more emulsifiers, e.g., from about 7 wt % to about 20 wt % of one or more emulsifiers. Preferably, the composition of the present invention includes about 9 wt % of one or more emulsifiers. Suitable emulsifiers that can be used in the composition of the present invention include, for example, sorbitans, alkoxylated fatty alcohols, alkylpolyglycosides, soaps, alkyl sulfates, monoalkyl and diallyl phosphates, alkyl sulphonates, acyl isothionates, and the like, and combinations thereof. Preferred emulsifiers include emulsifying waxes, and combinations thereof. In particularly preferred embodiments, the compositions can include about 6 wt % to about 10 wt % emulsifying wax.

The compositions of the present invention includes an oily phase, e.g., from about 5 wt % to about 50 wt % oily phase (e.g., from about 11 wt % to about 15 wt % oily phase). An exemplary composition of the present invention includes about 12 wt % oily phase. The oily phase preferably includes one or more pharmaceutically acceptable oil components suitable for topical administration such as, e.g., fatty acids, fatty alcohols, fatty acid esters, silicones, and the like, and combinations thereof. Preferably, the components of the oily phase have emollient or skin softening properties. Preferred oily phase components include, e.g., cetostearyl alcohol, oleic acid, caprylic capric triglyceride, and the like, and combinations thereof. In particularly preferred embodiments, the compositions can include about 10 wt % or less cetostearyl alcohol, about 1 wt % to about 3 wt % oleic acid, and/or about 10 wt % to about 12 wt % caprylic capric triglyceride.

The composition of the present invention also can include one or more additional active ingredients, including pharmaceutically active and cosmetically active ingredients, which can be water insoluble or only slightly soluble in water. Suitable additional active ingredients can include, e.g., anti-inflammatory agents, analgesics, anti-histamines, antiinfective agents (e.g., anti-viral, anti-fungal agents, anti-mycotic agents, anti-bacterial agents, components such as dibasic sodium phosphate heptahydrate and the like), antiacne agents, anti-psoriasis agents, wound healing agents, anti-wrinkle agents, skin rejuvenating agents, anti-pigmentation agents, anti-proliferative agents, growth factors, cytoxic agents, chemotherapeutic agents, and the like, and combinations thereof. Such ingredients, if included, will typically be present in relatively amounts of about 0.5 wt % or less.

If desired, the composition of the present invention can include one or more additional suitable corticosteroids, preferably in low doses. Exemplary additional corticosteroids can include commercially available or known corticosteroids such as, e.g., alcometasone, clocortolone, dexamethasone, hydrocortisone, hydrocortisone 21-acetate, prednisone, hydrocortisone 17-valerate, hydrocortisone 17-butyrate, betamethasone valerate, triamcinolone acetonide, flucinonide, desonide, flucinolone acetonide, dexamethasone, dexamethasone 21-phosphate, prednisolone, prednisolone 21-phosphate, haloprednone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone, flumethasone pivalate, flunisolide acetate, flucortolone, fluorometholone, fluperolone acetate, fluprednisolone, fluprednisolone valerate, meprednisone, methyl prednisolone, paramethasone acetate, prednisolamate, prednival, triamcinolone, triamcinolone hexacetonide, cortivazol, formocortal, nivazol, methylprednisone, and the like, and combinations thereof.

The composition of the present invention also can include other well-known pharmaceutically and/or cosmetically acceptable additives, such as, e.g., antioxidants, pH adjusting agents, chelating agents, preservative agents, occlusive agents, emollients, thickeners, solubilizing agents, tonicity agents, penetration enhancing or modifying agents, crystallization inhibiting agents, anti-irritants, and the like, and combinations thereof.

Suitable pH adjusting agents, which can be used in the composition of the present invention can include, for example, malic acid, lactic acid, citric acid, glycolic acid, benzoic acid, ascorbic acid adipic acid, glycines, phosphoric acid, calcium hydroxides, magnesium aluminometasilicates, buffer systems, and the like, and combinations thereof. If desired, one or more pH adjusting agents can be added to maintain desired pH levels.

Suitable chelating agents, which can be used in the composition of the present invention can include, for example, ethylenediaminetetraacetic acid (EDTA), EDTA derivatives and salts, and the like, and combinations thereof.

Suitable preservative agents, which can be used in the composition of the present invention can include, for example, benzyl alcohol, alkanols, disodium EDTA (ethylenediamine tetraacetate), EDTA salts, EDTA fatty acid conjugates, isothiazolinone, parabens (e.g., methylparaben and propylparaben), glycols, sorbates, diazolindinyl urea, and the like, and combinations thereof. In a preferred embodiment, compositions of the present invention can comprise about 1% to about 3% benzyl alcohol.

Suitable occlusive agents, which can be used in the composition of the present invention and can be incorporated in the oily phase of the composition of the present invention, can include, for example, petrolatum, mineral oil, beeswax, silicone oil, lanolin and oil-soluble lanolin derivatives, saturated and unsaturated fatty alcohols such as behenyl alcohol, hydrocarbons such as squalane, animal and vegetable oils (e.g., almond oil, peanut oil, wheat germ oil, linseed oil, jojoba oil, apricot pit oil, walnut oil, palm nut oil, pistachio nut oil, sesame seed oil, rapeseed oil, cade oil, corn oil, peach pit oil, poppyseed oil, pine oil, castor oil, soybean oil, avocado oil, safflower oil, coconut oil, hazelnut oil, olive oil, grape seed oil, sunflower seed oil), and the like, and combinations thereof. Some occlusive agents also can serve as emollients.

Suitable emollients, which can be used in the composition of the present invention include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof, and the like, and combinations thereof.

Suitable thickening agents, which can be used in the composition of the present invention can include, for example non-ionic water-soluble polymers such as, e.g., hydroxyethylcellulose (commercially available under the Trademark Natrosol® 250 or 350), cationic water-soluble polymers such as Polyquat 37 (commercially available under the Trademark Synthalen® CN), fatty alcohols, fatty acids and alkali salts thereof, and the like, and combinations thereof. It will be appreciated that some thickening agents also can be used as gelling agents.

Suitable solubilizing agents, which can be used in the composition of the present invention, can include, e.g., complex-forming solubilizer citric acid, ethylenediaminetetraacetate, sodium meta-phosphate, succinic acid, cyclodextrin, polyvinylpyrrolidone, diethylammonium-ortho-benzoate, or micell-forming solubilizers such as tweens and spans (e.g., Tween 80), and the like, and combinations thereof. Other suitable solubilizers can include, e.g., polyoxyethylene sorbitan fatty acid ester, polyoxyethylene n-alkyl ethers, n-alkyl amine n-oxides, poloxamers, organic solvents, phospholipids, cyclodextrins, and the like, and combinations thereof.

Suitable penetration enhancing or penetration modifying agents can include, e.g., dimethylsulfoxide (DMSO), dimethyl formamide (DMF), allantoin, urazole, Ndimethylacetamide (DMA), decylmethylsulfoxide (C₁₀MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, glycerin, hyaluronic acid, transcutol, and the like, and combinations thereof. Certain oil components (e.g., certain vegetable oils such as, e.g., safflower oil, cottonseed oil and corn oil) also can exhibit penetration enhancing or penetration modifying properties.

Suitable anti-irritants can include, e.g., aloe vera, chamomile, alpha-bisabolol, cola nitida extract, green tea extract, tea tree oil, licoric extract, allantoin, caffeine or other xanthines, glycyrrhizic acid and derivatives thereof, and the like, and combinations thereof.

The present invention additionally provides a method of producing a topical cream composition, which preferably includes mixing an active phase with an oily phase; combining a water phase with the mixture of oil and active phases, and homogenizing the phases to produce a substantially uniform dispersion; optionally adjusting the pH to obtain a pH of from about 4.0 to about 5.5; and, optionally, further diluting with water, to produce the composition. The active phase preferably includes a polyol and from about 0.05 wt % to less than 0.1 wt % mometasone furoate; the oily phase preferably includes a fatty acid, a fatty alcohol, a fatty acid ester, or a combination thereof; and the water phase preferably water and one or more gelling agents. The composition prepared in accordance with the method of the present invention preferably includes at least about 20 wt % water. In a particularly preferred embodiment, the method of the present invention includes preparing a water phase that includes water and one or more gelling agents; preparing an active phase that includes a polyol and about 0.075 wt % mometasone furoate; preparing an oily phase that includes a fatty acid, a fatty alcohol, and a fatty acid ester; mixing the active phase with the oily phase; combining the water phase with the mixed active and oily phases and homogenizing the phases; adjusting the pH to a pH of from about 4.0 to about 5.5; and adding sufficient purified water (qs as needed) to produce a composition that includes at least about 20 wt % water. Preferably, at least a substantial portion of the water in the composition of the present invention is bound water. More preferably, substantially all of the water in the composition of the present invention is bound water. One skilled in the art will appreciate that additional components, including but not limited to additional active ingredients, can optionally be included in the appropriate phase of preparation.

The composition of the present invention can be formulated in various dosage forms suitable for topical, transdermal, or buccal administration such as, for example, a foam, an aerosol, a spray, a gel, a cream, a lotion, an ointment, a suspension, an emulsion, a paste, a solution, and the like. The composition of the present invention is preferably formulated as a topical cream.

The present invention further provides a method for treating a corticosteroid-responsive disease or condition associated with a patient's skin, which method includes topically applying to the patient (e.g., on the patient's skin at or near the affected area) a therapeutically effective amount of a mometasone furoate composition of the present invention. Preferably, the disease or condition is a corticosteroid-responsive dermatosis. In a preferred embodiment, the disease or condition is an atopic dermatitis or a type of eczema.

The method of the present invention can be used for treating pediatric patients, e.g., pediatric patients under 2 years of age, under 18 months of age, under 1 year of age, or under 6 months of age.

In accordance with the method of the present invention, the composition can be safely applied to a relatively large percentage of the patient's total skin surface area (e.g., greater than about 20% of the patient's total skin surface area), even in pediatric patients under 2 years of age, with a reduction in toxicity and/or side effects otherwise exhibited by Elocon® Cream. For instance, the composition can be applied in any amount sufficient to contact up to about 80% of the patient's total skin surface area, e.g., about 20% to about 80%, about 20% to about 70%, about 20% to about 60%, about 20% to about 50%, about 20% to about 40%, or about 20% to about 30%.

In accordance with the method of the present invention, the patient can be treated over a relatively long period of time with a reduction in toxicity and/or side effects otherwise exhibited by Elocon® Cream. For instance, the composition of the present invention can be topically applied to the patient at least once per day for more than 3 weeks, e.g., about 3 weeks to about 6 months, about 3 weeks to about 5 months, about 3 weeks to about 4 months, about 3 weeks to about 3 months, about 3 weeks to about 2 months, or about 3 weeks to about 6 weeks.

The following examples further illustrate the invention but, of course, should not be construed as in any way limiting its scope.

### Example 1

This example illustrates a method of preparing an exemplary composition of the present invention.

100 grams of an aqueous cream composition containing 0.075% mometasone furoate are prepared by the following process.

The water phase is prepared first: Xanthan gum, and carbomer 940 are dispersed in purified water. Next, dibasic sodium phosphate heptahydrate is mixed into the dispersion. Emulsifying wax and benzyl alcohol are added to the dispersion and heated.

To prepare the active solution, mometasone furoate USP is dissolved in heated propylene glycol.

Next, the oily phase is prepared: Oleic acid, cetostearyl alcohol, and caprylic capric triglyceride are combined and mixed.

The active solution and the oily phase are added to the water phase.

The resulting emulsion is cooled. Emulsion pH is checked and adjusted with phosphoric acid as needed. Purified water is added to the emulsion to reach 100% (q.s.). The components of the exemplary composition (Formula A) are listed in Table 1.

TABLE 1
0.075% Mometasone Furoate - Exemplary Formula A
Formula B (Comparative)

| Ingredients | Formulation (wt %) |
|---|---|
| Mometasone Furoate | 0.075 |
| Xanthan Gum | 0.2 |
| Carbomer 940 | 0.3 |
| Dibasic Sodium Phosphate Heptahydrate | 0.2 |
| Emulsifying Wax | 8.0 |
| Benzyl Alcohol | 1.0 |
| Propylene Glycol | 15.0 |
| Cetostearyl Alcohol | 2.0 |
| Oleic Acid | 1.2 |
| Caprylic-Capric Triglyceride | 12.0 |
| Phosphoric Acid | Q.S. (About 0.05) |
| Purified Water | Q.S. (About 60.0) |

### Example 2

This example illustrates the high level of bound water in the formulations of the present invention.

The presence of bound (entrapped) water in Formula A, prepared as described in Example 1 above, is determined by DSC measurements performed in the endothermic scanning mode by controlled heating of previously frozen samples. The DSC curve expected to result from analysis of Formula A is characterized by an asymmetric broad peak at −6.30° C. The enthalpy change (ΔH) associated with a thermal transition (the total energy), is evaluated by integrating the area of this peak. Enthalpy (ΔH) of free water melting is −324 J/g. The actual enthalpy change demonstrated for Formula A is significantly smaller than −162 J/g, the predicted ΔH of a composition such as Formula A, which contains about 50% water. The temperature and enthalpy values indicate that the vast majority of the water in Formula A exists as bound water.

### Example 3

This example illustrates comparative mometasone furoate 0.1% composition (Formula B). The components of comparative Formula B are listed in Table 2.

TABLE 2
0.1% Mometasone Furoate - Formula B
Formula B (Comparative)

| Ingredients | Formulation (wt %) |
|---|---|
| Mometasone Furoate, USP | 0.1 |
| Phosphoric Acid | 0.525 |
| Purified Water | 52.675 |
| Xanthan Gum | 0.2 |
| Carbomer 940 | 0.3 |
| Dibasic Sodium Phosphate Heptahydrate | 1.0 |
| Emulsifying Wax (Polawax) | 8.0 |
| Benzyl Alcohol | 1.0 |
| Propylene Glycol | 15.0 |
| Cetostearyl Alcohol | 7.0 |
| Oleic Acid | 1.2 |
| Caprylic-Capric Triglyceride | 12.0 |

### Example 4

This example illustrates the comparison of Formula B relative to Elocon 0.1% cream composition.

A comparative synthetic membrane (Nyaflo membrane; Ethanol:Water (40:60) v/v receptor) penetration test was performed using Formula B (MMO21) relative to Elocon 8 0.1% cream (3NGF401).

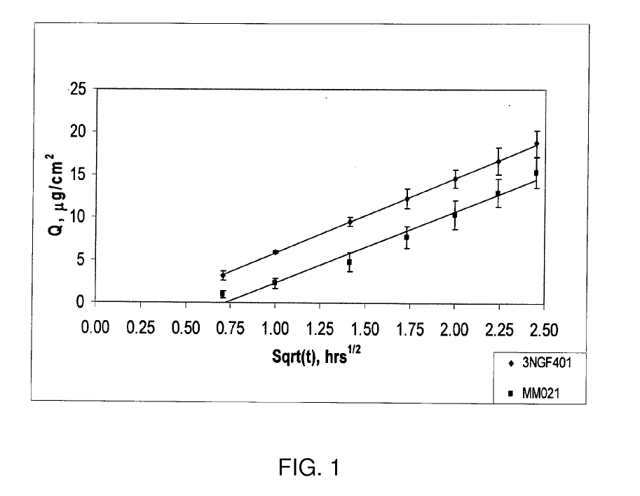

The results of the test show, as presented in FIG. 1, that the penetration of the active pharmaceutical ingredient from Formula B exhibits a similar penetration relative to the active pharmaceutical ingredient from the Elocon 0.1% composition.

### Example 5

This example illustrates an exemplary 0.075% mometasone furoate cream composition of the present invention (Formula C). The components of exemplary Formula C are listed in Table 3.

TABLE 3
0.075% Mometasone Furoate Cream - Formula C
Formula B (Comparative)

| Ingredients | Formulation (wt %) |
|---|---|
| Mometasone Furoate | 0.075 |
| Xanthan Gum | 0.2 |
| Carbomer 940 | 0.3 |
| Dibasic Sodium Phosphate Heptahydrate | 1.0 |
| Emulsifying Wax (Polawax) | 8.0 |
| Benzyl Alcohol | 1.0 |
| Propylene Glycol | 15.0 |
| Cetostearyl Alcohol | 7.0 |
| Oleic Acid | 1.2 |
| Caprylic-Capric Triglyceride | 12.0 |
| Phosphoric Acid | 0.525 |
| Purified Water | 53.7 |

### Example 6

This example illustrates the reduced potential for systemic toxicity exhibited by Formula C of the present invention relative to Formula B.

A comparative synthetic membrane (Nyaflo® membrane; Ethanol:Water (40:60) v/v receptor) penetration test was performed using Formula C (M11D0202, invention) relative to Formula B (M11D0203, comparative).

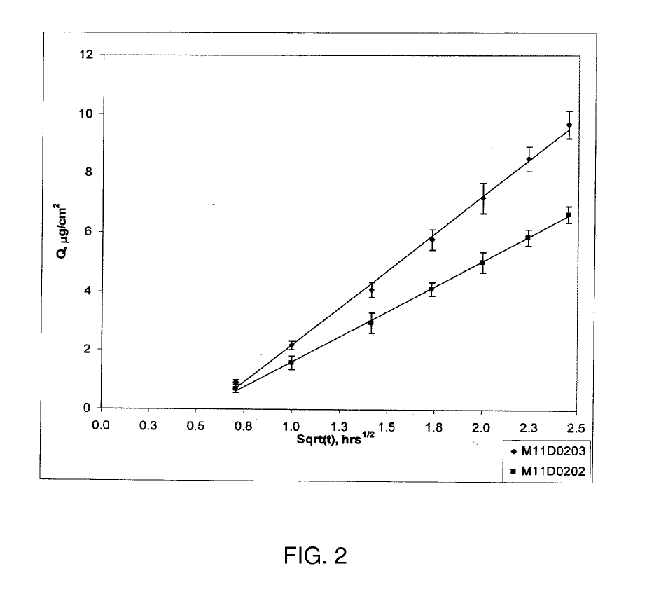

The results of the test show, as presented in FIG. 2, that Formula C exhibits reduced penetration relative to Formula B. Hence, the reduced penetration improves the safety of the 0.075% cream composition by reducing the toxicity of the composition.

### Example 7

This example compares the effect of the exemplary formulation of the present invention, Formula A mometasone furoate cream (0.075%, see Example 1), with Formula B mometasone furoate cream (0.1%, see Example 2) and Elocon® 0.1% cream on 12-O-tetradecanoylphorbol-13-acetate (TPA, Sigma) induced skin inflammation response in female Imprinting Control Region (ICR) mice.

Female ICR mice (40) were divided into 6 groups and the dorsal skin was treated with the following compositions (100 μl volume each) once daily for 10 days and 30 minutes prior to treatment with TPA every other day:

| Group | Components |
|---|---|
| Group 1 | none (untreated) |
| Group 2 | mometasone cream 0% (placebo) |
| | 0 μg TPA (acetone vehicle alone) |
| Group 3 | 5 μg TPA (8.5 nmol in acetone)* |
| Group 4 | mometasone cream 0.075% |
| | 5 μg TPA (in acetone) |
| Group 5 | mometasone cream 0.1% |
| | 5 μg TPA (in acetone) |
| Group 6 | Elocon ® 0.1% cream |
| | 5 μg TPA (in acetone) |

*mice were treated with 5 μg TPA every other day

A skin biopsy (3 cm×3 cm) was performed on each mouse and the effect of each composition on scores of dermal inflammatory cell infiltration and epidermal hyperplasia following inducement by administration of TPA to the external skin was determined. The scoring used for dermal inflammatory cell infiltration represents the percentage of tissue in the section involved in focal, multifocal or diffuse distribution of lesions (0 not present (0%), 1=slight (0-10%), 2=mild (11-20%), 3=moderate (21-40%), and 4 marked (41-100%)). The scoring used for epidermal hyperplasia represents the increase in the thickness of the epidermal layers (thickness of the epithelium from basal layer to stratum corneum) in treated animals compared to control animals (0=not present (0), 1=slight (2×control), 2 mild (3×control), 3=moderate (4×control), and 4=marked (5×control)).

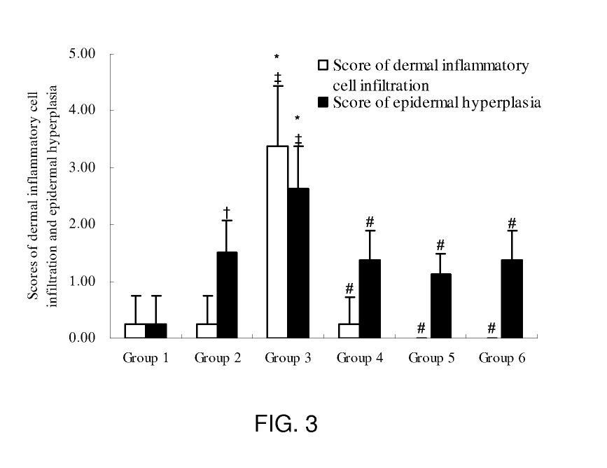

The results of the histopathological analysis test are summarized in FIG. 3. Mometasone cream 0.075% (Group 6) significantly reduced mean histopathology score of dermal inflammatory cells infiltration and epidermal hyperplasia induced by 5 μg (8.5 nmol) TPA (Group 3). There were no significant differences between the dermal therapeutic effects (anti-inflammatory and antiproliferative effects) of mometasone cream 0.075% (Group 6) and Elocon® 0.1% cream (Group 4). The therapeutic effects of mometasone 0.075% (Group 6) and mometasone cream 0.1% (Group 5) were comparable.

The results of the histopathological analysis test show that the mometasone cream 0.075% composition of the invention significantly reduced scores of dermal inflammatory cell infiltration and epidermal hyperplasia induced by 5 ug (8.5 nmol) TPA compared to placebo. In addition, there were no significant differences between therapeutic effects of mometasone cream 0.075% of the invention and Elocon® 0.1% cream and the therapeutic effects of mometasone cream 0.075% of the invention and mometasone cream 0.1% were comparable.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cream composition comprising from about 0.05 wt % to less than 0.1 wt % mometasone furoate in a pharmaceutically acceptable vehicle.

2. The composition of claim 1, further comprising an oily phase and at least about 20 wt % water.

3. The composition of claim 1, comprising from about 0.075 wt % to less than 0.1 wt % mometasone furoate.

4. The composition of claim 1, comprising about 0.075 wt % mometasone furoate.

5. The composition of claim 1, comprising from about 30 wt % water to about 65 wt % water.

6. The composition of claim 1, comprising about 60 wt % water.

7. The composition of claim 1, wherein the vehicle further comprises a polyol and a gelling agent.

8. The composition of claim 7, comprising from about 12 wt % to about 30 wt % of polyol.

9. The composition of claim 7, wherein the polyol is propylene glycol.

10. The composition of claim 7, comprising from 0.01 wt % to about 0.9 wt % of gelling agent.

11. The composition of claim 7, wherein the gelling agent is a polyacrylic acid.

12. The composition of claim 7, wherein the gelling agent is a carbomer.

13. The composition of claim 7, wherein the gelling agent comprises two or more gelling agents.

14. The composition of claim 13, wherein the two or more gelling agents are selected from carbomers, carboxymethylcellulose, ethylcellulose, gelatin, hydroxyethyl cellulose, hydroxypropyl cellulose, magnesium aluminum silicate, methylcellulose, poloxamers, polyvinyl alcohol, sodium alginate, alginic acid, tragacanth, acacia, bentonite, xanthan gum, and combinations thereof.

15. The composition of claim 13, wherein the two or more gelling agents comprise xanthan gum and a carbomer.

16. The composition of claim 1, further comprising a pH adjusting agent.

17. The composition of claim 16, wherein the pH adjusting agent is a buffer.

18. The composition of claim 1, wherein the composition is an oil-in-water emulsion.

19. The composition of claim 1, further comprising from about 6 wt % to about 20 wt % of an emulsifier.

20. The composition of claim 19, wherein the emulsifier comprises an emulsifying wax, a sorbitan, an alkoxylated fatty alcohol, an alkylpolyglycoside, a soap, an alkyl sulfate, a monoalkyl phosphate, a dialkyl phosphate, an alkyl sulphonate, an acyl isethionate or a mixture thereof.

21. The composition of claim 19, wherein the emulsifier comprises an Cetostearyl Alcohol

22. The composition of claim 1, comprising from about 10 wt % to about 25 wt % oily phase.

23. The composition of claim 1, wherein the oily phase comprises a fatty acid, a fatty alcohol, a fatty acid ester or a mixture thereof.

24. A method for producing a topical cream composition comprising: mixing an active phase with an oily phase, wherein the active phase comprises a polyol and about 0.075% mometasone furoate, and the oily phase comprises a fatty acid, a fatty alcohol, and a fatty acid ester; combining a water phase with the mixture of oil and active phases and homogenizing the phases to produce a substantially uniform dispersion, wherein the water phase comprises water and one or more gelling agents; optionally adjusting the pH to obtain a pH of from about 4.0 to about 5.5; and optionally further diluting with water, wherein the composition comprises and at least about 20 wt % water.

25. A method for treating a corticosteroid-responsive disease or condition associated with a patient's skin, the method comprising topically applying to the patient a therapeutically effective amount of a composition comprising from about 0.05 to less than 0.1 wt % mometasone furoate in a pharmaceutically acceptable vehicle.

26. The method of claim 25, wherein the corticosteroid-responsive skin condition is selected from the group consisting of inflammation, hyperkeratotic dermatosis, corticosteroid-responsive dermatosis, eczema, and atopic dermatitis.

27. The method of claim 25, wherein the skin condition is a corticosteroid-responsive dermatosis.

28. The method of claim 25, wherein the skin condition is eczema.

29. The method of claim 25, wherein the skin condition is atopic dermatitis.

30. The method of claim 25, wherein the patient is under 2 years of age.

31. The method of claim 25, wherein the patient is under 18 months of age.

32. The method of claim 25, wherein the patient is under 1 year of age.

33. The method of claim 25, wherein the patient is under 6 months of age.

34. The method of claim 25, comprising applying the composition in an amount sufficient to contact up to about 80% of the patient's total skin surface area.

35. The method of claim 34, wherein the patient is under 2 years of age.

36. The method of claim 34, wherein the patient is under 18 months of age.

37. The method of claim 34, wherein the patient is under 1 year of age.

38. The method of claim 34, wherein the patient is under 6 months of age.

39. The method of claim 25, wherein the patient is treated at least once per day for more than 3 weeks.

40. The method of claim 25, wherein the patient is treated at least once per day for more than 6 weeks.

41. The method of claim 25, wherein the patient is treated at least once per day for more than 3 months.

42. The method of claim 25, wherein the patient is treated at least once per day for about 6 months.

43. The method of claim 25, wherein the composition further comprises an oily phase and at least about 20 wt % water.

44. The method of claim 25, wherein the composition comprises from about 0.075 wt % to less than 0.1 wt % mometasone furoate.

45. The method of claim 25, wherein the composition comprises about 0.075 wt % mometasone furoate.
